(19) United States
(12) Patent Application Publication
(10) Pub. No.: US 2026/0199570 A1
(43) Pub. Date: Jul. 16, 2026

(54) VIBRATORY MYOFASCIAL DECOMPRESSION APPARATUS

(71) Applicants: Kyle Stewart, Encinitas, CA (US); Arizona Board of Regents on behalf of Arizona State University, Scottsdale, AZ (US)

(72) Inventors: Kendall Stewart, Chandler, AZ (US); Kyle Stewart, Encinitas, CA (US)

(21) Appl. No.: 19/561,196

(22) Filed: Mar. 9, 2026

Related U.S. Application Data
(62) Division of application No. 17/496,969, filed on Oct. 8, 2021.
(60) Provisional application No. 63/091,262, filed on Oct. 13, 2020.

(51) Int. Cl. A61M 1/08; A61H 9/00; A61H 23/04

(52) U.S. Cl. CPC A61M 1/08; A61H 9/0057; A61H 23/04; A61M 2205/3553; A61M 2205/8275

(57) ABSTRACT

A vibratory myofascial decompression apparatus includes a cup and at least one electrically operated vibration element positioned external to the cup. The cup includes a cup body defining an internal cavity and a lip that defines an opening into the internal cavity, with the lip being configured to contact a skin surface of a user. A housing engages an exterior of the cup body. The cup also includes an exhaust port positioned inside the internal cavity configured to permit air to be withdrawn from the internal cavity. The at least one electrically operated vibration element is coupled with the cup to permit vibration to be transmitted through the cup body and the lip to the skin of the user.

### STATEMENT OF RELATED APPLICATION(S)

This application is a divisional of U.S. patent application Ser. No. 17/496,969 filed on Oct. 8, 2021, which claims priority to U.S. Provisional Patent Application No. 63/091,262 filed on Oct. 13, 2020, wherein the entire disclosure of the foregoing applications are hereby incorporated by reference herein.

### TECHNICAL FIELD

The technology of the disclosure relates to myofascial decompression (also known as cupping) and vibration useful for therapeutic treatment of tissues of a user.

### BACKGROUND

Myofascial decompression (“MFD”), also known as cupping, is a technique that uses suction to create a vacuum that is applied to the skin and subcutaneous tissues. The vacuum is created by placing a cup device on a user's skin, and removing air from within a cup device, in order to decrease pressure within a cavity of the cup. Application of localized subatmospheric pressure to the skin causes the separation of the skin and subcutaneous tissues that is effective to break up scar tissue, increase mobility in various muscle groups and joints, and release stiffness. As such, the use of myofascial decompression is used to decrease pain in various muscle groups and joints within the body, to increase the health of a user, and/or to rehabilitate injuries. Often MFD is useful in the treatment of injuries caused from overuse such as bursitis, tendonitis, tendinosis, and other myofascial pain syndromes, such as, pain in the lower back, mid-back, neck, elbow, and shoulder.

MFD is often used widely by athletes and other physically active individuals who participate in various physical activities, exercises, or strenuous outdoor interactions, by which physical overreach may be encountered. Accordingly, these individuals often suffer from soft tissue damage which can lead to chronic pain, long-standing injuries, and other ailments if not properly treated. MFD may be used to enable a faster recovery from workout and to increase the user's overall physical well-being. Through the proper treatment, cups can be placed on the user's skin, to create suction that will effectively target fascia, or fibrous connective tissue that envelops and separates muscles and organs throughout the soft tissue structure in the body. The size and number of cups to be used may vary depending on the site(s) to be treated, as well as the size and shape of the user.

As a separate mode of treatment, therapists and medical professionals will often vibrate the skin of an individual in order to help loosen muscles, tissue, and increase blood flow. Vibration often allows for the release of tension in muscles and tissue in an area of a chronic injury to promote quicker healing. Additionally, vibration therapy can improve muscular strength, increase power development, increase kinesthetic awareness, decrease muscle soreness, increase range of motion, and increase blood flow under the skin. However, standard decompressors and vibration tools have limited penetration depth and ability to cling to tissue.

### SUMMARY

Exemplary aspects disclosed herein include a vibratory myofascial decompression (“MFD”) apparatus that is configured to provide relief or treatment to the soft tissue of a user's body. The vibratory MFD apparatus includes a cavity-defining cup configured to create a seal between a section of the user's body and the atmospheric air pressure conditions outside of the cup. The creation of subatmospheric conditions within the cup is configured to separate the skin and tissue underneath in order to promote healing of local tissues that may be injured or causing pain to a user. The cup further includes one or more electrically operated vibration elements that are positioned external to the cup, and are coupled with the cup to permit vibration to be transmitted through the cup to the skin of a user. The vibration of the sealed off muscles and skin is enabled to improve blood flow to the section of the body to which the cup is applied to provide therapeutic benefits.

In this regard, in exemplary aspects, the vibratory myofascial decompression apparatus includes a cup and at least one electrically operated vibration element positioned external to the cup. The cup includes a cup body defining an internal cavity, and a lip defining an opening into the internal cavity, with the lip being configured to contact a skin surface of a user. The cup also includes an exhaust port positioned inside the internal cavity configured to permit air to be withdrawn from the internal cavity. The at least one electrically operated vibration element is coupled with the cup to permit vibration to be transmitted through the cup body and the lip to the skin of the user.

In certain embodiments, the at least one electrically operated vibration element is configured to be coupled to a housing. The housing is configured to hold the cup and removably engage an exterior of the cup body. In certain embodiments, the housing may be configured to engage cups of different sizes to provide a modular device. The combination of vibration and suction generated by cupping can help with recovery from musculoskeletal injury and enhance the time between a next training session or physical therapy session.

In certain embodiments, a vibratory myofascial decompression system comprises a plurality of cups, a plurality of electrically operated vibration elements coupled with the plurality of cups to permit vibrations to be transmitted through the cup body and lip of each cup of the plurality of cups, and a controller configured to control operation of the plurality of electrically operated vibration elements. According to such an embodiment, each cup of the plurality of cups comprises a cup body defining an internal cavity, a lip configured to contact a skin surface of a user, and an exhaust port positioned inside the internal cavity configured to permit air to be withdrawn from the internal cavity.

Those skilled in the art will appreciate the scope of the present disclosure and realize additional aspects thereof after reading the following detailed description of the preferred embodiments in association with the accompanying drawing figures.

### BRIEF DESCRIPTION OF THE DRAWING FIGURES

The accompanying drawing figures incorporated in and forming a part of this specification illustrate several aspects of the disclosure, and together with the description serve to explain the principles of the disclosure.

FIG. 1 depicts an exemplary cup that includes an internal cavity that includes a lip to contact a section of a user's body, and an exhaust port to permit removal of air from the internal cavity to establish subatmospheric conditions, wherein the cup is configured to apply suction to the section of the user's body and transmit vibrations produced by electrically operated vibration elements;

FIG. 2 depicts an exemplary interface supporting a multiple electrically operated vibration elements arranged external to the cup;

FIG. 3 depicts an exemplary housing adapted to retain and secure the cup inside a housing cavity;

FIG. 4 depicts the external surface of the housing, wherein the housing is configured to be secured to the cup via a securing device and a set of fasteners;

FIG. 5 depicts the external surface of the housing, with the cup secured via a securing device and positioned such that the exhaust port permits removal of air from within the internal cavity of the cup; and

FIG. 6 depicts the internal cavity of the housing that includes the cup positioned within the housing.

FIG. 7 is a schematic illustrating interconnections between components of a vibratory myofascial decompression system including multiple vibratory decompression cups and a controller.

FIG. 8 is a schematic diagram of a generalized representation of a computer system that can be utilized as, or included in a component of, a vibratory myofascial decompression system as disclosed herein.

### DETAILED DESCRIPTION

The embodiments set forth below represent the necessary information to enable those skilled in the art to practice the embodiments and illustrate the best mode of practicing the embodiments. Upon reading the following description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present disclosure. As used herein, the term “and/or” includes any and all combinations of one or more of the associated listed items.

Relative terms such as “below” or “above” or “upper” or “lower” or “horizontal” or “vertical” may be used herein to describe a relationship of one element, layer, or region to another element, layer, or region as illustrated in the Figures. It will be understood that these terms and those discussed above are intended to encompass different orientations of the device in addition to the orientation depicted in the Figures.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises,” “comprising,” “includes,” and/or “including” when used herein specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

Exemplary aspects disclosed herein include a vibratory myofascial decompression (“MFD”) apparatus that is configured to provide relief or treatment to the soft tissue of a user's body. The vibratory MFD apparatus includes a cavity-defining cup configured to create a seal between a section of the user's body and the atmospheric air pressure conditions outside of the cup. Removal of air from the cup creates subatmospheric pressure conditions within the cup. The suction created by these conditions is configured to separate the skin and underlying tissue in order to assist with healing the section of the body which may be in pain or chronically injured. The cup further includes one or more vibration elements that are configured to transmit vibrations through the cup body and lip to the skin and underlying tissues proximate to the cup. The vibration of the skin and tissues may improve blood flow to the section of the body that is treated to assist with the healing process.

An exemplary vibratory myofascial decompression apparatus includes a cup having a cup body defining an internal cavity. The cup also includes a lip that bounds an opening of the internal cavity and that is configured to contact a skin surface of a user. The cup also includes an exhaust port positioned inside the internal cavity configured to permit air to be withdrawn from the internal cavity. The vibratory myofascial decompression apparatus also includes at least one electrically operated vibration element positioned external to the cup. The at least one electrically operated vibration element is also coupled with the cup to permit vibration to be transmitted through the cup body and the lip to the skin of the user.

Vibrations generated by one or more electrically operated vibration elements are transferred through the cup body to the lip of the cup, and transferred to the skin and underlying tissue of the user. Each of the electrically operated vibration elements are configured to be coupled to a housing. The housing is configured to engage an exterior of the cup body. In certain embodiments, the housing is configured to be removed from the cup, and may be used with cups of different sizes, resulting in a modular device wherein the vibration housing may be selectively engaged with cups of different sizes. The combination of vibration along with the suction generated by cupping can help with recovery from musculoskeletal injury, enhance user comfort, and/or reduce the time required before the next training session.

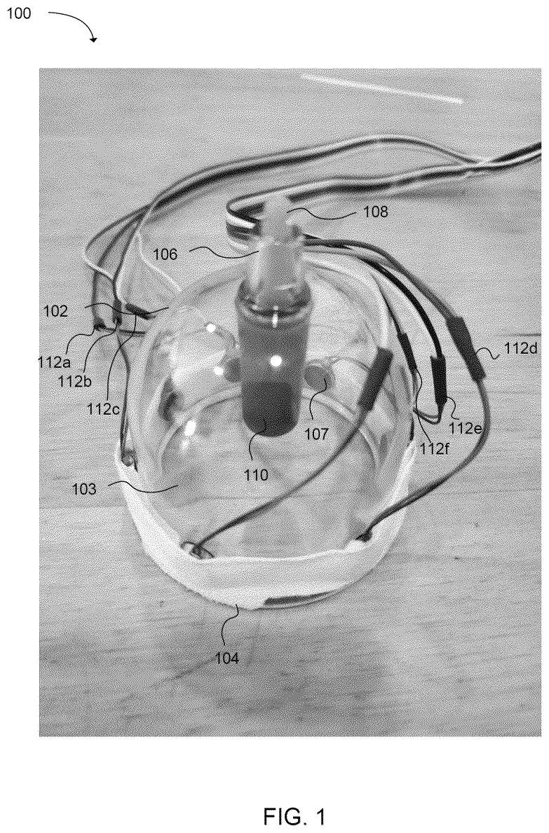

FIG. 1 depicts an exemplary cup device 100 that includes a cup 102 having an internal cavity 103 and including a lip 104 to contact a section of a user's body 103. The cup can be placed on any suitable area of the user's body requiring treatment. The body of the cup 102 may be of a generally cylindrical shape. In some examples, the cup can be of different sizes, depending on the type of therapy to be provided, or depending on the section of the user's body to which the cup will be applied. For example, larger areas of the body such as thighs, calves, back and chest are prone to chronic pain caused by various injuries, and may benefit from use of a cup 102 that is of a larger size in order to more effectively facilitate the therapy or treatment being provided. In the instance that the areas that are needed to be treated are smaller, or in need of a more targeted therapy or treatment, the size of the cup 102 can be smaller in order to further assist with the required treatment or therapy.

The lip 104 is configured to make an air-tight seal with the section of the user's skin. The lip 104, upon contacting the section of the user's body, is able to create a seal and a subsequent vacuum around the section of the body contacted by the lip 104. The seal is further bound by an opening of the internal cavity 103 of the cup 102. Upon the seal first being established by the lip 102, the internal cavity 103 is at a substantially atmospheric air pressure similar to the air pressure external to the cup 102. The existence of atmospheric air pressure conditions within the internal cavity 103 corresponds to a condition in which the cup 102 is not applying suction to the section of the user's body. Without being effectively suctioned on to the section of the user's body, the cup 102 can easily be moved, and can be considered inactive. When subatmospheric pressure conditions are established within the internal cavity 103, suction may be applied to a section of the user's skin.

The cup 102 also includes an exhaust port 106 to permit removal of air from the internal cavity 103 to establish a subatmospheric air pressure condition therein. The exhaust port 106 is positioned at the top of the cup 102 and is configured to release air pressure or receive air pressure through a valve 110, which may be embodied in a biased (e.g., spring-loaded) check valve that, when closed, enables a pressure differential to be maintained. In order for the cup 102 to create suction around the section of the user's body bounded by the lip 104, atmospheric air pressure is released from the internal cavity 103 of the cup 102. An air pressure releasing device (e.g., suction pump or the like) can be arranged to withdraw air from the internal cavity 103 to establish a subatmospheric air pressure condition therein, thereby applying suction on the section of the user's body that is contacted by the lip 104 of the cup 102. In certain embodiments, the valve 110 may include a spring biased moveable pin 108 positioned within the exhaust port 106.

In one example, the pin 108 can cooperate with one or more seating surfaces of the valve 110 in order to create a seal between the internal cavity 103 of the cup 102, and the atmospheric air pressure external to the cup. In some examples, a user can operate the pin 108 to enable release of a pressure differential and thereby permit the cup device 100 to be removed from the section of the user's body.

The cup device 100 can further comprise a plurality of electrically operated vibration elements 107 positioned around the external portion of the cup 102. Each of the electrically operated vibration elements 107 is configured to vibrate the body of the cup 102, in order to create a vibration effect at the section of the user's body that is contacted by the lip 104. The electrically operated vibration elements 107 can be configured to vibrate simultaneously or vibrate randomly at various positions around the body of the cup 102. The electrically operated vibration elements 107 can also be set at one or more frequencies in order to be adapted for various therapies or treatment regimens for different tissues. In some instances, the electrically operated vibration elements 107 can be manipulated by an operator or the user. The electrically operated vibration elements 107 can also be configured to vibrate in a pattern or after a predetermined time period. In some instances, a set of electrically operated vibration elements 107 could be selected to vibrate during a first time period, and a second set can be configured to vibrate during a second time period, wherein the first and second time periods may be non-overlapping or partially overlapping in character. The electrically operated vibration elements 107 can further include a set of wire leads 112a-112f that are configured to send one or more signals to each of the electrically operated vibration elements 107. In certain embodiments, each electrically operated vibration element 107 may be powered by a battery, by a power supply, or other power source operatively coupled with the wire leads 112a-112f. One or more potentiometers, variable resistors, or the like may be used to adjust amplitude and/or frequency of vibrations generated by the electrically operated vibration elements 107.

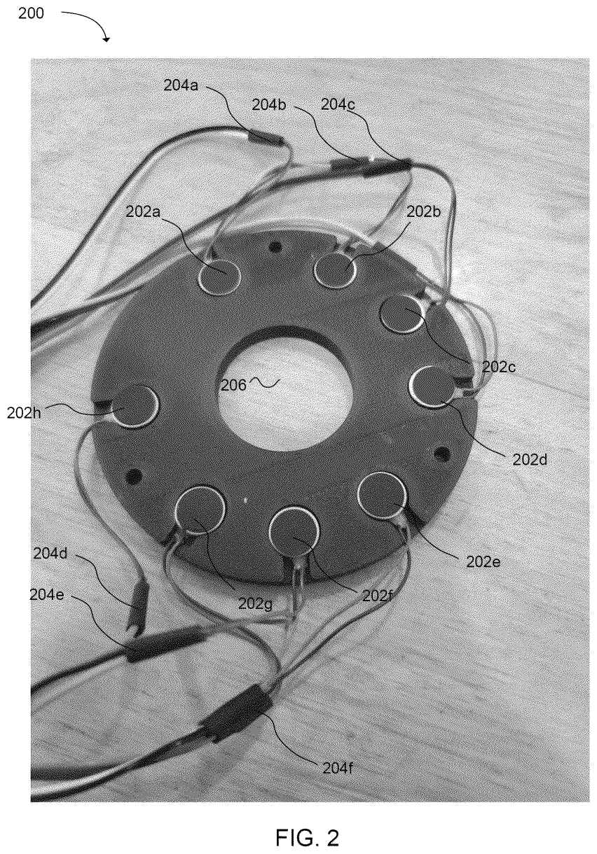

In one example, at least one electrically operated vibration element may comprise electrically operated vibration elements 202a-202h and comprise a disc motor that is attached to an interface element 200, as shown in FIG. 2. In some instances, the electrically operated vibration element can be an offset motor or a combination of both disc motors and offset motors. Each of the electrically operated vibration elements are configured to be coupled with a second end of the set of wire leads 112a-112f, which may be joined by an interface element 200 to adjoining wire leads 204a-204e. The adjoining wire leads 204a-204e may be configured to attach to a second set of electrically operated vibration elements 202a-202h. Accordingly, the interface element 200 is configured to support at least one of the electrically operated vibration elements 202a-202h, and is positioned at a proximate end of the cup 102 that opposes a proximal end of the cup 102 where the lip 104 is positioned. The interface element 200 further defines an opening 206 in the body of the interface element 200 that provides access to the exhaust port 106.

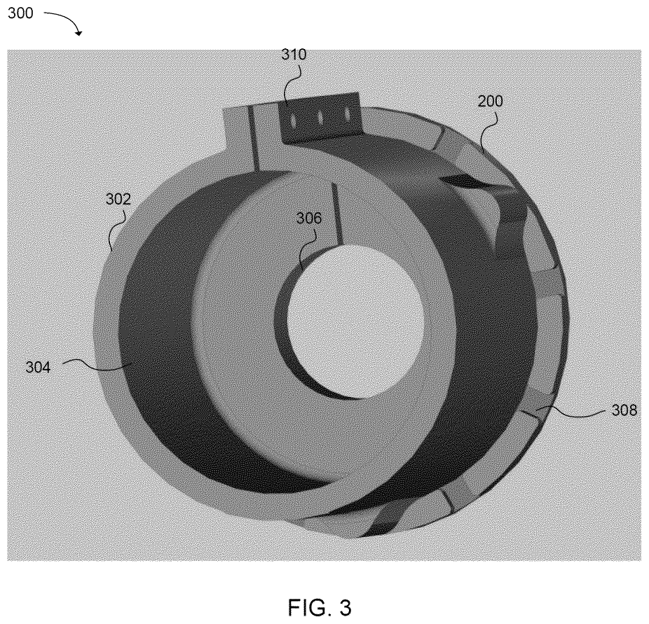

The interface element 200 is configured to be removably coupled with a housing 300, as depicted in FIG. 3. The interface 200 further comprises a set of inserts 308 that are configured to retain a second set of electrically operated vibration elements 202a-202h. As shown in FIG. 3, the second set of electrically operated vibration elements 202a-202h may be configured for insertion into the inserts 308, in order to transmit vibrations to the housing 300 that are propagated through the cup 102 to a section of the user's skin to which suction is applied by the cup 102. FIG. 3 depicts an exemplary housing 300 adapted to retain and secure the cup 102 inside a housing cavity 304. The housing 300 is positioned external to the body of the cup 102, and configured to engage an exterior of the body of the cup 102. Accordingly, at least a portion of the external portion 302 of the housing 300 comprises an annular shape in order to engage the external surface of the cup 102. The housing 300 is configured to enclose the cup body, and also secure the exterior of the cup body to the housing 300 via a securing device 310. Additionally, at least one of the plurality of electrically operated vibration elements 107 is coupled to the housing 300. The electrically operated vibration elements 107 are configured to vibrate the exterior of the cup body, to cause vibrations to travel through the body of the cup 102 to the section of the user's skin that is contacted by the lip 104. In one embodiment, at least one potentiometer is coupled with at least one electrically operated vibration element 107 to permit adjustment of at least one operating characteristic of the at least one electrically operated vibration element 107. The housing 300 further comprises a top opening 306 that is configured to allow the exhaust port 106 to protrude through the top of the housing 300. The protrusion of the exhaust port 106 allows the user or an operator to interact with the pin 108, in order to influence pressure conditions within the internal cavity 103 of the cup 102.

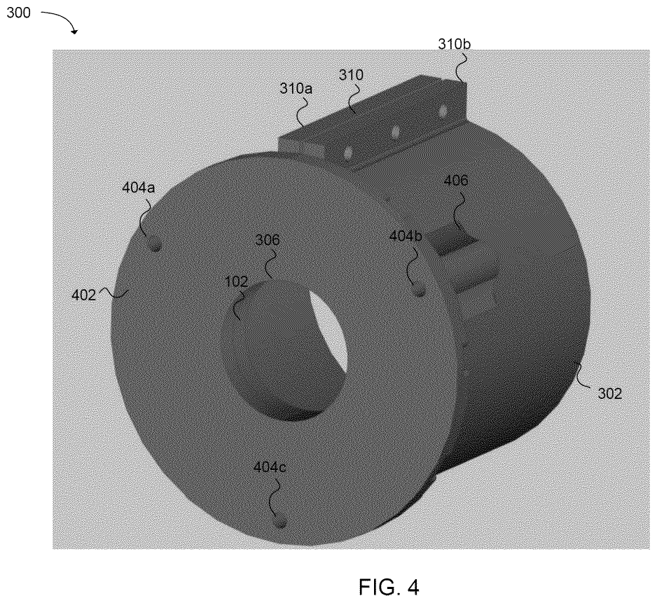

The housing 300 further includes a securing device 310 that is configured to be secured to the cup 102 inside of the internal cavity 304 of the housing 300. FIG. 4 depicts the external surface of the housing 300 wherein the cup is configured to be secured to the cup via the securing device 310. As shown, the securing device 310 protrudes from the annular body of the housing 300, and comprises two securing portions, including a first securing portion 310a and a second securing portion 310b. Each of the first 310a and second securing portions 310b are configured to receive one or more fasteners. Each of the fasteners is configured to be slidably attached through the first securing portion 310a, and through the second securing portion 310b. Each securing portion 310a-310b is configured to be tightly secured by the one or more fasteners, whereby adjustment of the securing portions 310a-310b may adjust tightness of the securing device 310.

In certain embodiments, the securing device 310 can comprise a ratcheting closure. The ratcheting closure can be configured to permit at least a portion of the housing 300 to be tightened around an outer perimeter of the body of the cup 102. The ratcheting closure can also be configured to manipulate the distances between each of the first securing portion 310a and the second securing portion 310b. As the ratcheting closure is tightened, distance between the first securing portion 310a and the second securing portion 310b will vary based on the degree by which a user or operator has tightened the housing 300 around the cup 102.

Additionally, the housing 300 comprises a top portion 402 that is secured to the top of the interface element 200, and to the body 302 of the housing 300. The top portion 402 is configured to receive a set of fasteners through one or more through holes 404a-404c that are configured to receive the set of fasteners in order to secure the top portion 402, and the interface element 200, to the body of the housing 302. The fasteners are configured to be secured to the body 302 via one or more fastening elements 406 that can be threaded to secure the fasteners received through the top through holes 404a-404c. The fastening element can be one or more of screws, bolts, or another fastening mechanism.

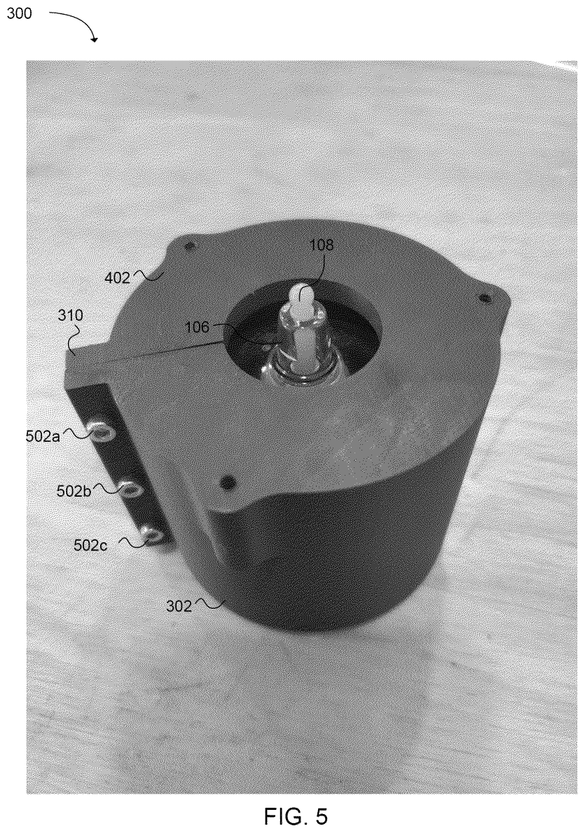

FIG. 5 depicts the external surface of the housing 300 wherein the cup 102 is secured via a securing device 310 and positioned such that the exhaust port 106 may selectively release air from within the internal cavity 103 of the cup 102. The cup 102 is configured to be secured by the one or more fasteners 502a-502c secured through the securing device 310. The cup 102 is configured to be contacted by the internal cavity 304 of the housing 300. As shown in FIG. 5, the housing 300 is configured to provide a top opening 306 registered with the exhaust port. An air removing (or air releasing) device is configured to cooperate with the exhaust port in order to effectively manipulate the air pressure conditions in the internal cavity 103 of the cup 102. An air removing device may comprise be one or more of an electric pump, a hand pump, a compressor, a machine, or another device that is configured to cooperate with the valve 110 to create a differential pressure condition between the internal cavity 103 and a surrounding environment.

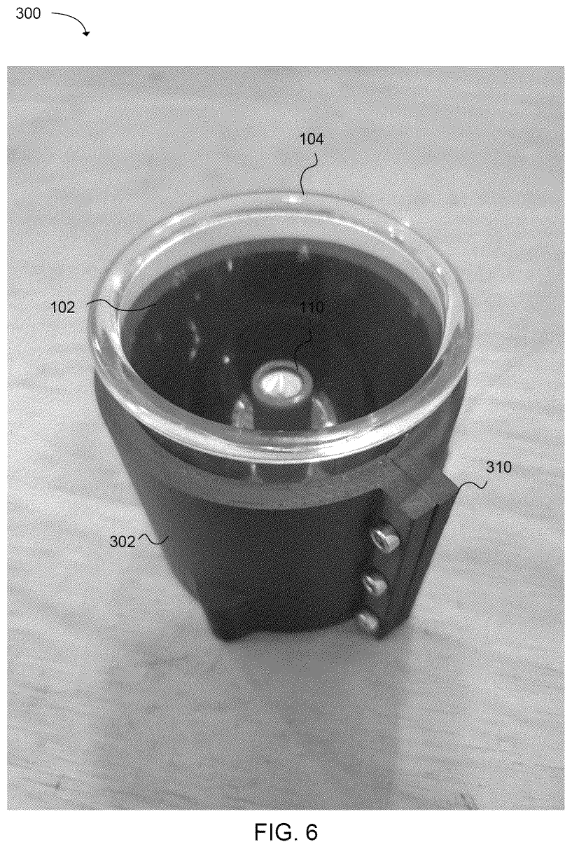

FIG. 6 depicts the internal cavity 304 of the housing 300 that includes the cup 102 positioned within the body 302 of the housing 300, with the cup 102 being operable to contact a section of the user's body. As shown, the cup 102 is configured to be secured within the housing 300, such that the cup 102 can be held by the body 302, and placed by the operator on a section of the user's body. The lip 104 of the cup 102 is placed on the section of the user's body that is targeted for treatment. Upon establishment of subatmospheric air pressure within the internal cavity 103 of the cup 102, the electrically operated vibration elements 107 can be configured to transmit vibrations through the cup body and lip 104 to the skin and underlying tissues of the user proximate to the cup 102. If a different sized cup 102 is desired, the securing device 310 can be loosened, and a new cup 102 can be placed within the internal cavity 304 of the housing 300. Once the new cup 102 has been positioned within the internal cavity 304 of the housing 300, the securing device 310 can be re-secured via the one or more fasteners 502a-502c and/or the ratcheting or clamping device.

In one aspect, the disclosure relates to a method for therapeutic treatment of a user utilizing a MFD apparatus as disclosed herein. Such a method includes placing the lip of the cup in contact with a skin surface of the user; withdrawing air from the internal cavity of the cup through the exhaust port to establish suction between the cup and the skin surface of the user; and supplying electric power to the electrically operated vibration element to cause vibration to be transferred through the cup body and the lip of the cup to the skin surface of the user.

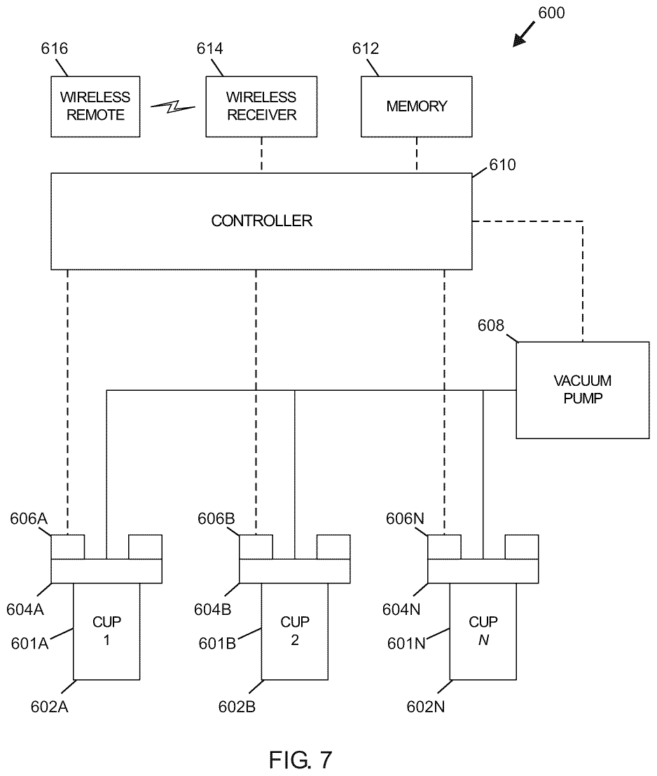

In one aspect, the disclosure is directed to a vibratory myofascial decompression system that comprises a plurality of cups, a plurality of electrically operated vibration elements, and a controller configured to control operation of the vibration elements and/or pressure conditions within the plurality of cups. An exemplary vibratory myofascial decompression system 600 is schematically illustrated in FIG. 7. The system 600 includes multiple cups 601A-601N (wherein N represents any suitable number greater than one) each having a lip 602A-602N along a proximal end thereof, and each having an interface element 604A-604N with one or more vibration elements 606A-606N along a distal end thereof. Each cup 601A-601N is operatively coupled with an air removal device such as a vacuum pump 606. The vibration elements 606A-606N and the vacuum pump 606 receive control signals from a controller 610 to enable pressure conditions in the cups 601A-601N and operation of the vibration elements 606A-606N to be controlled. A memory 612 associated with the controller 610 may be used to store one or more pre-defined or user-defined treatment regimens that may be selected for controlling parameters such as treatment duration, vibration intensity, vibration frequency, vibration pattern, subatmospheric pressure level, etc. A wireless receiver 614 operatively coupled with the controller 610 may be used to receive signals from a wireless remote control element 616 (e.g., an infrared or radio frequency remote control element, optionally embodied in a smartphone) to enable a therapist or user to wirelessly control vibratory myofascial decompression treatment parameters. In certain embodiments, the wireless receiver 614 and wireless remote control element 616 may be configured to communicate using any suitable wireless protocol or communication type, such as Bluetooth®, ZigBee®, or the like. In one example, the plurality of cups 601A-601N may be positioned along the length of a muscle or group of muscles to create a recovery chain can be created. A recovery chain can be created by positioning the plurality of cups 601A-601N in a series formation on the skin surface of the user across the length of a muscle or a muscle group.

In certain embodiments, the vibratory myofascial decompression system 600 of FIG. 7 may include a single cup 601A. In certain embodiment, the single cup 601A may be controlled wirelessly via a computing device such as a smartphone, using any suitable wireless protocol or communication type, such as Bluetooth®, ZigBee®, etc.

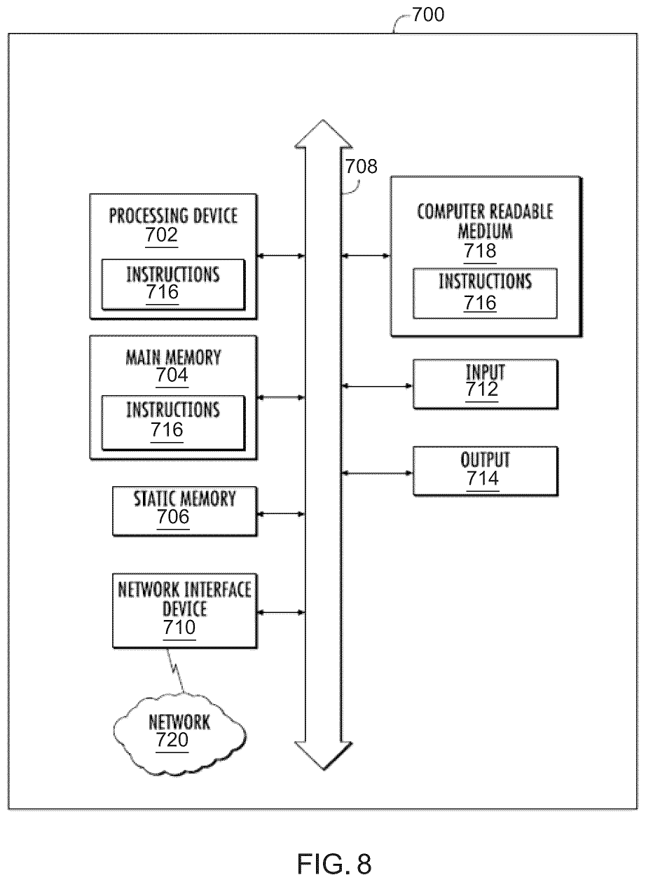

FIG. 8 is a schematic diagram of a generalized representation of a computer system (optionally embodied in a computing device, including but not limited to a mobile device such as a smartphone) that can be utilized as, or included in a component of, a vibratory myofascial decompression system as disclosed herein. In this regard, the computer system 700 is adapted to execute instructions from a computer-readable medium to perform these and/or any of the functions or processing described herein. The computer system 700 in FIG. 8 may include a set of instructions that may be executed to program and configure programmable digital circuits for controlling a vibratory myofascial decompression system. The computer system 700 may be connected (e.g., networked) to other machines in a LAN, an intranet, an extranet, or the Internet. While only a single device is illustrated, the term “device” shall also be taken to include any collection of devices that individually or jointly execute a set (or multiple sets) of instructions to perform any one or more of the methodologies discussed herein. The computer system 700 may be a circuit or circuits included in an electronic board card, such as a printed circuit board (PCB), a server, a personal computer, a desktop computer, a laptop computer, a personal digital assistant (PDA), a computing pad, a mobile device, or any other device, and may represent, for example, a server or a user's computer.

The computer system 700 in this embodiment includes a processing device or processor 702, a main memory 704 (e.g., read-only memory (ROM), flash memory, dynamic random access memory (DRAM), such as synchronous DRAM (SDRAM), etc.), and a static memory 706 (e.g., flash memory, static random access memory (SRAM), etc.), which may communicate with each other via a data bus 708. Alternatively, the processing device 702 may be connected to the main memory 704 and/or static memory 706 directly or via some other connectivity means. The processing device 702 may be a controller, and the main memory 704 or static memory 706 may be any type of memory.

The processing device 702 represents one or more general-purpose processing devices, such as a microprocessor, central processing unit, or the like. More particularly, the processing device 702 may be a complex instruction set computing (CISC) microprocessor, a reduced instruction set computing (RISC) microprocessor, a very long instruction word (VLIW) microprocessor, a processor implementing other instruction sets, or other processors implementing a combination of instruction sets. The processing device 702 is configured to execute processing logic in instructions for performing the operations and steps discussed herein.

The computer system 700 may further include a network interface device 710. The computer system 700 also may or may not include an input 712, configured to receive input and selections to be communicated to the computer system 700 when executing instructions. The computer system 700 also may or may not include an output 714, including but not limited to a display, a video display unit (e.g., a liquid crystal display (LCD) or a cathode ray tube (CRT)), an alphanumeric input device (e.g., a keyboard), and/or a cursor control device (e.g., a mouse).

The computer system 700 may or may not include a data storage device that includes instructions 716 stored in a computer readable medium 718. The instructions 716 may also reside, completely or at least partially, within the main memory 704 and/or within the processing device 702 during execution thereof by the computer system 700, the main memory 704 and the processing device 702 also constituting computer readable medium. The instructions 716 may further be transmitted or received over a network 720 via the network interface device 710.

While the computer readable medium 718 is shown in an embodiment to be a single medium, the term “computer-readable medium” should be taken to include a single medium or multiple media (e.g., a centralized or distributed database, and/or associated caches and servers) that store the one or more sets of instructions. The term “computer readable medium” shall also be taken to include any medium that is capable of storing, encoding, or carrying a set of instructions for execution by the processing device 702 and that cause the processing device 702 to perform any one or more of the methodologies of the embodiments disclosed herein. The term “computer readable medium” shall accordingly be taken to include, but not be limited to, solid-state memories, optical media, and magnetic media.

The embodiments disclosed herein include various steps. The steps of the embodiments disclosed herein may be executed or performed by hardware components or may be embodied in machine-executable instructions, which may be used to cause a general-purpose or special-purpose processor programmed with the instructions to perform the steps. Alternatively, the steps may be performed by a combination of hardware and software.

Unless otherwise expressly stated, it is in no way intended that any method set forth herein be construed as requiring that its steps be performed in a specific order. Accordingly, where a method claim does not actually recite an order to be followed by its steps, or it is not otherwise specifically stated in the claims or descriptions that the steps are to be limited to a specific order, it is in no way intended that any particular order be inferred.

It will be apparent to those skilled in the art that various modifications and variations can be made without departing from the spirit or scope of the invention. Since modifications, combinations, sub-combinations and variations of the disclosed embodiments incorporating the spirit and substance of the invention may occur to persons skilled in the art, the invention should be construed to include everything within the scope of the appended claims and their equivalents.

What is claimed is:

1. A vibratory myofascial decompression apparatus comprising:
a cup comprising:
a cup body defining an internal cavity,
a lip configured to contact a skin surface of a user, and
an exhaust port positioned inside the internal cavity configured to permit air to be withdrawn from the internal cavity;
a housing configured to engage an exterior of the cup body; and
at least one electrically operated vibration element coupled to the housing and positioned external to the cup;
wherein the at least one electrically operated vibration element is configured to vibrate the exterior of the cup body, to permit vibration to be transmitted through the cup body and the lip to the skin of the user.

2. The apparatus of claim 1, wherein the cup body comprises a generally cylindrical shape, and at least a portion of the housing comprises an annular shape.

3. The apparatus of claim 1, wherein the housing is configured to enclose the cup body and secure an exterior of the cup body to the housing via a securing device.

4. The apparatus of claim 3, wherein the securing device comprises a clamp configured to engage around the exterior of the cup body.

5. The apparatus of claim 1, wherein the housing comprises a ratcheting closure that permits at least a portion of the housing to be tightened around an outer perimeter of the cup body.

6. The apparatus of claim 1, further comprising an interface element supporting the at least one electrically operated vibration element, wherein the interface element is configured to be removably coupled with the housing.

7. The apparatus of claim 6, wherein the lip is positioned at a proximal end of the cup body, and the interface element is arranged proximate to a distal end of the cup body that opposes the proximal end.

8. The apparatus of claim 6, wherein the interface element defines an opening that is registered with the exhaust port.

9. The apparatus of claim 1, wherein the cup further comprises a check valve in fluid communication with the exhaust port.

10. The apparatus of claim 1, wherein the exhaust port comprises a spring biased pin configured to restrict air from entering the internal cavity when subatmospheric conditions are established in the internal cavity.

11. The apparatus of claim 1, wherein the at least one electrically operated vibration element comprises a disc motor, an offset motor, or a piezoelectric element.

12. The apparatus of claim 1, further comprising at least one potentiometer coupled with the at least one electrically operated vibration element to permit adjustment of at least one operating characteristic of the at least one electrically operated vibration element.

13. The apparatus of claim 1, wherein the exhaust port is configured to be removably attached to an air removing device by which air may be removed from the internal cavity to establish subatmospheric air pressure conditions in the internal cavity.

14. The apparatus of claim 1, wherein the at least one electrically operated vibration element comprises a plurality of electrically operated vibration elements.

15. A vibratory myofascial decompression system comprising:
a plurality of vibratory myofascial decompression apparatuses each according to the vibratory myofascial decompression apparatus of claim 1; and
a controller configured to control operation of the at least one electrically operated vibration element of each vibratory myofascial decompression apparatus of the plurality of vibratory myofascial decompression apparatuses, and configured to control pressure conditions within the internal cavity of the cup of each vibratory myofascial decompression apparatus of the plurality of vibratory myofascial decompression apparatuses.

16. The system of claim 15, wherein the controller is configured to receive signals from a wireless remote control element to permit at least one of (i) control of operation of the at least one electrically operated vibration element of each vibratory myofascial decompression apparatus, and (ii) control of pressure conditions within the internal cavity of the cup of each vibratory myofascial decompression apparatus.

17. The system of claim 15, wherein cups of the plurality of vibratory myofascial decompression apparatuses are configured to be positioned in a series formation on the skin surface of the user across a length of a muscle or muscle group.

18. A method for therapeutic treatment of a user utilizing at least one vibratory myofascial decompression apparatus according to claim 1, the method comprising:
for each vibratory myofascial decompression apparatus, placing the lip of the cup in contact with a skin surface of the user;
vibratory myofascial decompression apparatus, withdrawing air from the internal cavity of the cup through the exhaust port to establish suction between the cup and the skin surface of the user; and
vibratory myofascial decompression apparatus, supplying electric power to the at least one electrically operated vibration element to cause vibration to be transferred through the cup body and the lip of the cup to the skin surface of the user.

19. The method of claim 18, wherein the at least one vibratory myofascial decompression apparatus comprises a plurality of vibratory myofascial decompression apparatuses.
